(19) United States
(12) Patent Application Publication
(10) Pub. No.: US 2015/0038464 A1
(43) Pub. Date: Feb. 5, 2015

(54) METHODS OF INCREASING LIGHT RESPONSIVENESS IN A SUBJECT WITH RETINAL DEGENERATION

(71) Applicant: Ralph J. Jensen, Norwood, MA (US)

(72) Inventor: Ralph J. Jensen, Norwood, MA (US)

(73) Assignee: The United States Government as represented by the Department of Veterans Affairs, Washington, DC (US)

(21) Appl. No.: 14/380,618

(22) PCT Filed: May 17, 2012
(86) PCT No.: PCT/US2012/038432

§ 371 Date: Aug. 22, 2014

Related U.S. Application Data
(60) Provisional application No. 61/602,889, filed on Feb. 24, 2012.

(51) Int. Cl. A61K 31/675; A61K 45/06; A61K 31/4741

(52) U.S. Cl. CPC A61K 31/675; A61K 31/4741; A61K 45/06
USPC 514/89; 514/291

(57) ABSTRACT

Disclosed herein are methods of increasing retinal responsiveness to light in a subject, such as a subject with retinal degeneration. The disclosed methods include administering one or more compounds that decrease or inhibit γ-aminobutyric acid (GABA) signaling to a subject with retinal degeneration. In some embodiments, the methods include selecting a subject with retinal degeneration and administering a γ-aminobutyric acid C (GABA_{C}) receptor antagonist to the subject. In one example, the GABA_{C} receptor antagonist is (1,2,5,6-tetrahydropyridin-4-yl)methylphosphinic acid (TPMPA). In other embodiments, the methods include selecting a subject with retinal degeneration and administering a metabotropic glutamate receptor (mGluR) antagonist to the subject. In one example, the mGluR antagonist is a mGlu1 receptor antagonist (for example, JNJ16259685).

### CROSS REFERENCE TO RELATED APPLICATION

This claims the benefit of U.S. Provisional Application No. 61/602,889, filed Feb. 24, 2012, which is incorporated herein by reference in its entirety.

### ACKNOWLEDGMENT OF GOVERNMENT SUPPORT

This invention was made with government support under a Merit Review Grant awarded by the Department of Veterans Affairs. The government has certain rights in the invention.

### FIELD

This disclosure relates to methods of increasing retinal responsiveness to light in a subject, particularly a subject with retinal degeneration.

### BACKGROUND

Photoreceptor degeneration is a leading cause of blindness in people worldwide. Retinitis pigmentosa (RP) is one of the most common forms of retinal degeneration. RP is a heterogeneous group of retinal degenerations, leading first to night blindness, and subsequently progressive loss of peripheral and central vision. In age-related macular degeneration (AMD), the cells of the macula in particular degenerate, leading to loss of central vision and decreased visual acuity.

Treatment options for these conditions remain limited. Currently, therapeutic approaches are generally restricted to slowing down the degenerative process by sunlight protection and vitamin therapy, treating complications (such as cataract and macular edema), and helping patients to cope with the social and psychological impact of blindness.

### SUMMARY

Disclosed herein are methods of increasing retinal responsiveness to light in a subject, such as a subject with retinal degeneration. The disclosed methods include administering one or more compounds that decrease or inhibit retinal γ-aminobutyric acid (GABA) signaling in a subject with retinal degeneration. In some examples, an inhibitor of GABA signaling includes a compound that decreases or inhibits GABA receptor signaling (such as a GABA receptor antagonist) and/or a compound that decreases or inhibits GABA release from a neuron (such as a metabotropic glutamate receptor antagonist). In some embodiments, the methods include selecting a subject with retinal degeneration and administering a GABA_{C} receptor antagonist to the subject. In other embodiments, the methods include selecting a subject with retinal degeneration and administering a metabotropic glutamate receptor (mGluR) antagonist to the subject. In some embodiments, the methods further include measuring retinal responsiveness to light in the subject. In some examples, the retinal responsiveness to light in the subject is increased, for example as compared to a control. In one non-limiting example, the GABA_{C} receptor antagonist is (1,2,5,6-tetrahydropyridin-4-yl)methylphosphinic acid (TPMPA). In additional non-limiting examples, the mGluR antagonist is a mGlu1 receptor antagonist (for example, JNJ16259685).

The foregoing and other features of the disclosure will become more apparent from the following detailed description, which proceeds with reference to the accompanying figures.

### BRIEF DESCRIPTION OF THE DRAWINGS

FIG. 1 is a graph showing an exemplary drug-induced change in the intensity-response curve for a retinal ganglion cell (RGC). The maximum peak response (indicated by “A”) is the result of fit of data points. The dynamic operating range (indicated by “B”) is defined as the range of light intensity that elicits response between 10 and 90% of maximum peak response. Drug-induced change in light sensitivity (indicated by “C”) is determined by comparing the light intensity that evokes a half-maximum response before drug application with the light intensity that evokes the same response in the presence of the drug.

FIG. 2 is an intensity-response curve of an RGC of a P23H rat, taken before and after application of 100 μM TPMPA to the bathing solution. Values on the abscissa are the number of log units of attenuation in stimulus intensity from the maximal (8.5×10¹⁷ photons/cm²/s).

FIG. 3 is a series of plots showing TPMPA-induced change in light-sensitivity (FIG. 3A), maximum peak response (FIG. 3B), and dynamic operating range (FIG. 3C) of P23H rat RGCs. The lines connect individual RGCs before and after TPMPA treatment.

FIG. 4 is a series of plots showing TPMPA-induced change in light-sensitivity (FIG. 4A), maximum peak response (FIG. 4B), and dynamic operating range (FIG. 4C) of SD rat RGCs. The lines connect individual RGCs before and after TPMPA treatment.

FIG. 5 is a graph of intensity-response curves of an RGC of a SD rat, taken before and after application of 100 μM TPMPA to the bathing solution. Values on the abscissa are the number of log units of attenuation in stimulus intensity from the maximal (8.5×10¹⁷ photons/cm²/s).

### DETAILED DESCRIPTION

Individuals with retinal degeneration (for example, RP or AMD) have a higher threshold for electrical or light stimulation of retinal responses than individuals without retinal degeneration (Rizzo et al., Invest. Ophthalmol. Vis. Sci. 44:5355-5361, 2003; Gekeler et al., Invest. Ophthalmol. Vis. Sci. 47:4966-4974, 2006; Jensen and Rizzo, J. Neural Eng. 8:035002, 2011). One treatment option under development for retinal degeneration is the use of a retinal prosthesis to at least partially restore vision. Reducing the amount of stimulation required (decreasing the threshold or increasing the retinal responsiveness to light or electrical stimulation) is an important step to improve the safety of such devices and make them practical for long term use.

As disclosed herein, inhibition of retinal GABA signaling increases retinal responsiveness to light in a rat model of RP. Ocular administration of GABA_{C} or mGlu1 receptor antagonists, for example by intraocular administration (such as intravitreal injection), subconjunctival injection, or topical administration, presents a promising therapy for individuals with RP, AMD, or other retinal degenerations where response thresholds are decreased. The GABA_{C} and mGlu1 receptor antagonists may also be administered systemically (for example, intravenously or orally). In particular, although GABA_{C} receptors are expressed in the brain, they are most prominently expressed in the retina, for example in bipolar cells, retinal ganglion cells, horizontal cells, and photoreceptors. Thus, systemic administration of a GABA_{C} receptor antagonist (for example, intravenously or orally) may produce minimal effects outside the retina and may be feasible for increasing retinal responsiveness to light in a subject.

### I. ABBREVIATIONS

AMD age-related macular degeneration

ERG electroretinogram

GABA γ-aminobutyric acid

JNJ16259685 3,4-dihydro-2H-pyranol[2,3-b]quinolin-7-yl-(cis-4-methoxycyclohexyl)-methanone

mGlu1 metabotropic glutamate receptor type 1

mGluR metabotropic glutamate receptor

RGC retinal ganglion cell

RP retinitis pigmentosa

SD Sprague-Dawley rat

TPMPA (1,2,5,6-tetrahydropyridin-4-yl)methylphosphinic acid

### II. TERMS

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. As used herein, “comprises” means “includes.” Thus, “comprising A or B,” means “including A, B, or A and B,” without excluding additional elements. It is further to be understood that all base sizes or amino acid sizes, and all molecular weight or molecular mass values, given for nucleic acids or polypeptides are approximate, and are provided for description. All publications, patent applications, patents, and other references mentioned herein are incorporated by reference in their entirety for all purposes.

Although methods and materials similar or equivalent to those described herein can be used in the practice or testing of the present disclosure, suitable methods and materials are described below. In case of conflict, the present specification, including explanations of terms, will control. In addition, the materials, methods, and examples are illustrative only and not intended to be limiting.

In order to facilitate review of the various embodiments of the disclosure, the following explanations of specific terms are provided:

Age-related macular degeneration (AMD): A condition in which the cells of the macula (the central part of the retina) degenerate, resulting in loss of central visual acuity. AMD is the most common cause of irreversible loss of central vision and legal blindness in the elderly. It causes progressive damage to the macula, resulting in gradual loss of central vision. There are two forms, atrophic and neovascular macular degeneration. In atrophic degeneration (dry form), the tissues of the macula thin as photoreceptor cells disappear. There is currently no treatment for atrophic degeneration, though dietary supplements may help slow progression. In neovascular macular degeneration (wet form), abnormal blood vessels develop under the macula. These vessels may leak fluid and blood under the retina and eventually a mound of scar tissue develops under the retina. Central vision becomes washed out and loses detail, and straight lines may appear wavy. For neovascular macular degeneration there are some treatments available, including the use of medication injected directly into the eye (e.g., anti-VEGF therapy), laser therapy in combination with a targeting drug (e.g., photodynamic therapy) and brachytherapy. However, repeated treatments can cause complications leading to loss of vision.

Effective amount: A dose or quantity of a specified compound sufficient to induce a desired response or result, for example to inhibit advancement, or to cause regression of a disease or disorder, or which is capable of relieving one or more symptoms caused by the disease. The preparations disclosed herein are administered in effective amounts. In some examples, this can be the amount or dose of a disclosed GABA_{C} or mGlu1 receptor antagonist required to increase retinal responsiveness to light in a subject, such as a subject with a retinal degeneration. In one embodiment, a therapeutically effective amount is the amount that alone, or together with one or more additional therapeutic agents (such as additional agents for treating a retinal disorder), induces the desired response, such as increasing retinal responsiveness to light in the subject.

γ-Aminobutyric Acid C (GABA_{C}) Receptor: Also Known as GABA_{A}-Rho (GABA_{A}-ρ) receptor. The GABA_{C} receptor is a subclass of GABA_{A} receptors, which are ligand-gated chloride channels. GABA_{C} receptors are insensitive to bicuculline and baclofen and are not modulated by benzodiazepines and barbiturates (which are GABA_{A} receptor modulators). There are three GABA_{C} receptor subunits (ρ1 (GABRR1), ρ2 (GABRR2), and ρ3 (GABRR3)). The GABA_{C} receptor is formed by oligomerization of five subunits, either as a homo-pentamer or a hetero-pentamer.

Nucleic acid and protein sequences for GABA_{C} receptor subunits are publicly available. For example, GenBank Accession Nos. NM_{—}002042 and NM_{—}017291 disclose exemplary human and rat GABA_{C} receptor ρ1 subunit (GABRR1) nucleic acid sequences, respectively, and GenBank Accession Nos. NP_{—}002033 and NP_{—}058987 disclose exemplary human and rat GABA_{C} receptor ρ1 subunit (GABRR1) protein sequences, respectively. GenBank Accession Nos. NM_{—}002043 and NM_{—}017292 disclose exemplary human and rat GABA_{C} receptor ρ2 subunit (GABRR2) nucleic acid sequences, respectively, and GenBank Accession Nos. NP_{—}002034 and NP_{—}058988 disclose exemplary human and rat GABA_{C} receptor ρ2 subunit (GABRR2) protein sequences, respectively. GenBank Accession Nos. NM_{—}001105580 and NM_{—}138897 disclose exemplary human and rat GABA_{C} receptor ρ3 subunit (GABRR3) nucleic acid sequences, respectively, and GenBank Accession Nos. NP_{—}001099050 and NP_{—}620252 disclose exemplary human and rat GABA_{C} receptor ρ3 subunit (GABRR3) protein sequences, respectively. Each of these GenBank Accession Nos. are incorporated by reference as provided by GenBank on Feb. 24, 2012.

A GABA_{C} receptor antagonist is a compound that inhibits expression and/or activity of a GABA_{C} receptor. In some examples, a GABA_{C} receptor antagonist inhibits (for example, statistically significantly inhibits) activity and/or expression of a GABA_{C} receptor, and may also inhibit or stimulate expression and/or activity of GABA_{A} and/or GABA_{B} receptors. In other examples, a GABA_{C} receptor antagonist inhibits (for example, statistically significantly inhibits) expression and/or activity of GABA_{C} receptors, but not GABA_{A} or GABA_{B} receptors (for example, is a selective GABA_{C} receptor antagonist). A GABA_{C} receptor antagonist can include a small molecule inhibitor, a polypeptide, an antisense compound, or an antibody.

Metabotropic glutamate receptor (mGluR): The metabotropic glutamate receptors are a family of G protein-coupled receptors that have been divided into 3 groups on the basis of sequence homology, putative signal transduction mechanisms, and pharmacologic properties. Group I includes mGlu1 and mGlu5 and these receptors have been shown to activate phospholipase C. Group II includes mGlu2 and mGlu3, and Group III includes mGlu4, mGlu6, mGlu7 and mGlu8. Group II and III receptors are linked to the inhibition of the cyclic AMP cascade but differ in their agonist selectivity. L-glutamate is the major excitatory neurotransmitter in the central nervous system and activates both ionotropic and metabotropic glutamate receptors. Glutamatergic neurotransmission is involved in most aspects of normal brain function and can be perturbed in many neuropathologic conditions. mGlu1 is also known as GRM1; GLUR1; mGluR1; GPRC1A; and mGluR1A.

Nucleic acid and protein sequences for mGlu1 receptors are publicly available. For example, GenBank Accession Nos. NM_{—}00114329 and NM_{—}000838 disclose exemplary human mGlu1 receptor nucleic acid sequences, and GenBank Accession Nos. NP_{—}001107801 and NP_{—}000829 disclose exemplary human mGlu1 receptor protein sequences. GenBank Accession Nos. NM_{—}017011 and NM_{—}001114330 disclose exemplary rat mGlu1 receptor nucleic acid sequences, and GenBank Accession Nos. NP_{—}058707 and NP_{—}001107802 disclose exemplary rat mGlu1 receptor protein sequences. Each of these GenBank Accession Nos. are incorporated by reference as provided by GenBank on May 15, 2012.

A mGlu1 receptor antagonist is a compound that inhibits expression and/or activity of a mGlu1 receptor. In some examples, a mGlu1 receptor antagonist inhibits (for example, statistically significantly inhibits) activity and/or expression of a mGlu1 receptor, and may also inhibit or stimulate expression and/or activity of one or more mGlu receptor subtypes. In other examples, a mGlu1 receptor antagonist inhibits (for example, statistically significantly inhibits) expression and/or activity of mGlu1 receptor receptors, but not mGlu5 receptors (for example, is a selective mGlu1 antagonist). In some examples, a mGlu1 receptor antagonist inhibitors or decreases release of GABA from a neuron, such as a retinal neuron (see, e.g., Vigh et al., Neuron 46:469-482, 2005). A mGlu1 receptor antagonist can include a small molecule inhibitor, a polypeptide, an antisense compound, or an antibody.

Pharmaceutically acceptable carriers: The pharmaceutically acceptable carriers useful in this disclosure are conventional. Remington: The Science and Practice of Pharmacy, The University of the Sciences in Philadelphia, Editor, Lippincott, Williams, & Wilkins, Philadelphia, Pa., 21^{st} Edition (2005), describes compositions and formulations suitable for pharmaceutical delivery of the compounds disclosed herein. In general, the nature of the carrier will depend on the particular mode of administration being employed.

Retinal degeneration: Deterioration of the retina, including progressive death of the photoreceptor cells of the retina or associated structures (such as retinal pigment epithelium). Retinal degeneration includes diseases or conditions such as retinitis pigmentosa, cone-rod dystrophy, macular degeneration (such as age-related macular degeneration and Stargardt-like macular degeneration), and maculopathies.

Retinal ganglion cell (RGC): A neuron located in the ganglion cell layer of the retina. RGCs receive neural inputs from amacrine cells and/or bipolar cells (which themselves receive neural input from photoreceptor cells). The axons of RGCs form the optic nerve, which transmits information from the retina to the brain.

Retinal responsiveness to light: The ability of one or more cells of the retina to respond to light (directly or indirectly), for example by producing an electrical signal and/or perception of a visual stimulus by a subject. Retinal response to light can be measured by detecting number, size, and/or frequency of electrical signals from the retina, for example by direct retinal recording (in vitro or in vivo), electroretinogram, or measuring visual evoked responses. Retinal response to light can also be measured by reporting of detection of a visual stimulus by a subject, for example wherein the subject closes a switch or presses a button when a visual stimulus is seen.

Retinitis pigmentosa (RP): A group of inherited retinal disorders that eventually lead to partial or complete blindness, characterized by progressive loss of photoreceptor cell function. Symptoms of RP include progressive peripheral vision loss and night vision problems (nyctalopia) that can eventually lead to central vision loss. RP is caused by mutations is over 100 different genes, and is both genotypically and phenotypically heterogeneous. Approximately 30% of RP cases are caused by a mutation in the rhodopsin gene. The pathophysiology of RP predominantly includes cell death of rod photoreceptors; however, some forms affect cone photoreceptors or the retinal pigment epithelium (RPE). Typical clinical manifestations include bone spicules, optic nerve waxy pallor, atrophy of the RPE in the mid periphery of the retina, retinal arteriolar attenuation, bull's eye maculopathy, and peripheral retinal atrophy.

Subject: Living multi-cellular vertebrate organisms, a category that includes both human and non-human mammals.

### III. METHODS OF INCREASING RETINAL RESPONSIVENESS TO LIGHT

Disclosed herein are methods of increasing retinal responsiveness to light in a subject, such as a subject with retinal degeneration. The methods include administering a compound that decreases or inhibits GABA signaling (such as a compound that decreases or inhibits retinal GABA signaling) to a subject with retinal degeneration. In some examples, the methods include administering to the subject a compound that decreases or inhibits (for example, selectively decreases or inhibits) GABA signaling in the retina of a subject with retinal degeneration. An inhibitor of GABA signaling is any compound that reduces or inhibits an aspect of transmission of a signal mediated by GABA, for example by one or more neurons. In some examples, an inhibitor of GABA signaling inhibits or decreases GABA receptor activity (such as a GABA receptor antagonist, for example a GABA_{C} receptor antagonist). In other examples, an inhibitor of GABA signaling inhibits or decreases release of GABA by a neuron (for example a mGluR antagonist, such as a mGlu1 receptor antagonist). In some examples, an inhibitor of GABA signaling inhibits or decreases GABA signaling at one or more retinal cells, including, but not limited to RGCs, amacrine cells, bipolar cells, or horizontal cells. In some examples, the inhibitor of GABA signaling decreases retinal GABA receptor activity. In other examples, the inhibitor of GABA signaling decreases or inhibits release of GABA by a retinal neuron.

In some embodiments, the methods include selecting a subject (such as a human subject) with retinal degeneration and administering a GABA_{C} receptor antagonist to the subject. In other embodiments, the methods include selecting a subject (such as a human subject) with retinal degeneration and administering a mGluR antagonist (such as a mGlu1 receptor antagonist) to the subject. In particular embodiments, the retinal degeneration is in a particular portion of the retina, for example in the macula and/or the fovea (as in macular degeneration) or in the peripheral retina (as in RP). In some embodiments, the methods further include measuring retinal responsiveness to light in the subject. In some examples, the retinal responsiveness to light in the subject is increased, for example as compared to a control.

In some examples, the methods include selecting and treating a subject with a retinal pathology (such as RP, AMD, or other disorder arising in the retina or associated structures). In particular examples, the subject does not have a refractive disorder of the eye (such as myopia). In other examples, the subject has a refractive disorder and a retinal disorder. In some examples, the subject does not have a cognitive deficit or memory impairment (such as dementia or Alzheimer's disease) or does not have a cognitive deficit or memory impairment associated with a disorder such as AIDS or schizophrenia. In other examples, the subject does not have a chronic neurological disorder of the central nervous system, such as Huntington disease, amyotrophic lateral sclerosis, Parkinson disease, migraine, epilepsy, or depression. In some examples, the methods include inhibiting GABA signaling selectively in the eye or the retina of the subject, for example, inhibiting or decreasing GABA signaling in the eye or retina of the subject, but not inhibiting or decreasing GABA signaling outside of the eye.

Methods for measuring or assessing visual function, retinal function (such as responsiveness to light stimulation), or retinal structure in a subject are well known to one of skill in the art. See, e.g., Federman et al. Retina and Vitreous, Textbook of Ophthalmology, Vol. 9, Mosby-Yearbook, Europe, Ltd., 1994; Kanski, Clinical Ophthalmology, A Systematic Approach, 3^{rd} Edition, Butterworth-Heinemann, Ltd., 1994. In some examples, methods for measuring or assessing retinal response to light include detecting an electrical response of the retina to a light stimulus. In some examples, the response is detected by measuring an electroretinogram (ERG; for example full-field ERG, multifocal ERG, or ERG photostress test), visual evoked potential, or optokinetic nystagmus (see, e.g., Wester et al., Invest. Ophthalmol. Vis. Sci. 48:4542-4548, 2007). In other examples, retinal response to light is measured by directly detecting retinal response (for example by use of a microelectrode at the retinal surface). In further examples, retinal responsiveness to light can be measured by exposing the subject to light stimuli (for example one or more pulses of light) and asking the subject to report detection of the stimulus, for example orally or by pushing a button, closing a switch, or other similar reporting means. The intensity of the light stimulus can be increased or decreased to measure a light sensitivity threshold. For example, the retinal sensitivity to light is measured by determining the intensity threshold, which is the minimum luminance of a test spot required to produce a visual sensation (perception) or electrical response of the retina. This can be measured by placing a subject in a dark or light room and increasing the luminance of a test spot until the subject reports its presence or an electrical response is detected. The test spot can be a focal spot of light directed at a fixed location on the retina, for example the fovea or a location in the peripheral retina.

In some embodiments of the disclosed methods, increasing retinal responsiveness to light in a subject includes an increase in one or more measures of retinal response, for example about a 10% to a 100-fold or more increase (such as at least about a 10% 20%, 30%, 40%, 50%, 60%, 70%, 80%, 90%, 1.5-fold, 2-fold, 3-fold, 5-fold, 10-fold, 20-fold, 30-fold, 40-fold, 50-fold, 60-fold, 70-fold, 80-fold, 90-fold, 95-fold, 100-fold increase, or more) in the subject as compared to a control. In some examples, an increase in retinal responsiveness to light includes an increase in the number, size (amplitude), dynamic range, and/or frequency of an electrical response by the retina to one or more light stimuli as compared to a control. In other examples, an increase in retinal responsiveness to light also includes a decreased threshold for stimulation of an electrical response to a light stimulus, for example, a detectable response or a response of a particular magnitude is evoked at a lower light intensity as compared to a control. In further examples, an increase in retinal responsiveness to light includes a decreased threshold for stimulation of a visible signal in response to a light stimulus, for example, a visible signal that is detectable (reported) by the subject is evoked at a lower light intensity as compared to a control. In a particular example, the change is detected in the intensity threshold. In yet other embodiments, more global measurements of visual function are used, such as an improvement in visual acuity (for example, measured on a Snellen chart), at least a partial restoration of a visual field deficit (for example, measured on a Humphrey Field Analyzer of Nidek microperimeter), such as a decrease in the size of a central visual field deficit of the type seen in macular degeneration or a peripheral visual field deficit as seen in RP, improvement in contrast sensitivity, or improvement in flicker sensitivity.

The control can be any suitable control against which to compare visual function or retinal function of a subject (such as retinal responsiveness to light). In some embodiments, the control is a reference value or ranges of values. For example, in some examples, the reference value is derived from the average values obtained from a group of subjects with a retinal degeneration (such as the same or a different retinal disorder as the subject), for example, an untreated subject or a subject treated with vehicle alone. In other examples, the control is obtained from the same subject, for example, a subject with retinal degeneration prior to treatment. In further examples, the reference value can be derived from the average values obtained from a group of normal control subjects (for example, subjects without a retinal degeneration).

In some embodiments, the methods include selecting a subject with retinal degeneration. In some examples, the subject is a mammalian subject (such as a human subject or a primate or rodent subject). A subject with retinal degeneration can be identified utilizing standard diagnostic methods, including but not limited to, measuring or assessing visual function, retinal function, and/or retinal structure of the subject, such as visual acuity, visual field, ERG, Amsler grid, fundus examination, color vision, fluorescein angiography, optical coherence tomography, or a combination of two or more thereof. In some examples, a retinal degeneration includes retinitis pigmentosa (RP), Usher syndrome, Stargardt's disease, cone-rod dystrophy, Leber congenital amaurosis, a retinopathy (such as diabetic retinopathy), a maculopathy (for example, age-related macular degeneration (AMD), Stargardt-like macular degeneration, vitelliform macular dystrophy (Best disease), Malattia Leventinese (Doyne's honeycomb retinal dystrophy), diabetic maculopathy, occult macular dystrophy, or cellophane maculopathy), congenital stationary night blindness, degenerative myopia, or damage associated with laser therapy (for example, grid, focal, or panretinal), including photodynamic therapy.

A. GABA_{C} Receptor Antagonists

In some embodiments, the GABA_{C} receptor antagonists of use in the disclosed methods are small organic molecule antagonists. In some examples, a GABA_{C} receptor antagonist includes 3-amino-propyl-n-butyl-phosphinic acid (CGP36742 or SGS742), 3-aminopropyl(methyl)phosphinic acid (SKF-97541), (Z)-3-[(aminoiminomethyl)thio]prop-2-enoic acid (ZAPA), or imidazole-4-acetic acid (I4AA). In other examples, a GABA_{C} receptor antagonist includes TPMPA, (3-aminopropyl)methylphosphinic acid, 3-aminopropylphosphinic acid, 3-aminopropylphosphonic acid, (±)-cis-(3-aminocyclopentyl)butylphosphinic acid, (3-aminocyclopentyl)methylphosphinic acid, 3-(aminomethyl)-1-oxo-1-hydroxy-phospholane (3-AMOHP), 3-(guanido)-1-oxo-1-hydroxy-phopholane (3-GOHP), (S)-(4-aminocyclopent-1-enyl)butylphosphinic acid, 2-aminoethyl methylphosphonate (2-AEMP), (piperidin-4-yl)methylphosphinic acid (P4MPA), piperidin-4-ylseleninic acid (SEPI), or (aminocyclopentane)methylphosphinic acid (ACPBuPA). See e.g., Chebib et al. (Neuropharmacology 52: 779-787, 2007), Ng et al. (Future Med. Chem. 3(2): 197-209, 2011), Xie et al. (Molecular Pharmacology 80(6): 965-978, 2011), Chebib et al. (J. Pharmacol. Exp. Ther. 328:448-457, 2009), Gavande et al. (Med. Chem. Lett. 2:11-16, 2011), U.S. Pat. App. Publ. Nos. 2006/0142249 and 2008/0032950; each of which is incorporated by reference herein. In one particular example, the GABA_{C} receptor antagonist is TPMPA.

In some examples, a GABA_{C} receptor antagonist has a structure represented by:

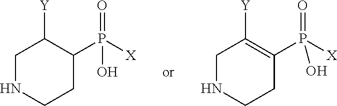

wherein X represents halogen, an alkyl group (optionally substituted with a halogen), or a hydroxyalkyl group and Y represents hydrogen, a halogen, or an alkyl, alkenyl, alkynyl, or acyl group (optionally substituted with halogen, nitrile, or NO₂).

In other examples, the GABA_{C} receptor antagonist has a structure represented by:

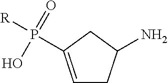

wherein R is methyl, ethyl, propyl, isopropyl, butyl, pentyl, neo-pentyl or cyclohexyl.

See, e.g., U.S. Pat. Publ. Nos. 2006/0142249 and 2008/0032950, both incorporated herein by reference.

In other embodiments, the GABA_{C} receptor antagonist is an antisense compound. Any type of antisense compound that specifically targets and regulates expression of a GABA_{C} receptor (such as a GABA_{C} receptor subunit) is contemplated for use. Methods of designing, preparing and using GABA_{C} receptor antisense compounds are within the abilities of one of skill in the art, for example, utilizing publicly available GABA_{C} receptor sequences. Antisense compounds specifically targeting GABA_{C} receptor can be prepared by designing compounds that are complementary to a GABA_{C} receptor nucleotide sequence, such as a GABA_{C} receptor ρ1, ρ2, and/or ρ3 mRNA sequence. Antisense compounds need not be 100% complementary to the target nucleic acid molecule to specifically hybridize and regulate expression the target gene. For example, the antisense compound, or antisense strand of the compound if a double-stranded compound, can be at least 75%, at least 80%, at least 85%, at least 90%, at least 95%, at least 99%, or 100% complementary to the selected GABA_{C} receptor nucleic acid sequence, such as about 20-25 contiguous nucleotides of a GABA_{C} receptor nucleic acid (for example, one or more GABA_{C} receptor subunits). Particular examples of GABA_{C} receptor nucleic acid sequences are provided above. Exemplary GABA_{C} receptor antisense compounds are commercially available (for example, from Santa Cruz Biotechnologies (Santa Cruz, Calif.); or Thermo Scientific Dharmacon (Lafayette, Colo.)). Methods of screening antisense compounds for specificity are well known in the art.

In other embodiments, the GABA_{C} receptor antagonist is an antibody. Any type of antisense compound that specifically binds and regulates activity of a GABA_{C} receptor (such as a GABA_{C} receptor subunit) is contemplated for use. One of ordinary skill in the art can readily generate antibodies which specifically bind to a GABA_{C} receptor (such as a GABA_{C} receptor subunit). These antibodies can be monoclonal or polyclonal. They can be chimeric or humanized. Any functional fragment or derivative of an antibody can be used including Fab, Fab′, Fab2, Fab′2, and single chain variable regions. So long as the fragment or derivative retains specificity of binding for the GABA_{C} receptor it can be used in the methods provided herein. Antibodies can be tested for specificity of binding by comparing binding to appropriate antigen (e.g., a GABA_{C} receptor subunit or portion thereof) to binding to irrelevant antigen or antigen mixture under a given set of conditions. If the antibody binds to appropriate antigen at least 2, at least 5, at least 7, or 10 times more than to irrelevant antigen or antigen mixture, then it is considered to be specific. Exemplary GABA_{C} receptor antibodies are commercially available (for example, from Santa Cruz Biotechnologies (Santa Cruz, Calif.); or Abcam (Cambridge, Mass.)).

In some embodiments, the GABA_{C} receptor antagonist is a selective GABA_{C} receptor antagonist, for example, a compound that inhibits activity or expression of a GABA_{C} receptor, but does not inhibit (for example, does not statistically significantly inhibit) activity or expression of other GABA receptors (such as GABA_{A} or GABA_{B} receptors). In some embodiments, a GABA_{C} receptor antagonist inhibits (for example, statistically significantly inhibits) expression and/or activity of GABA_{C} receptors, but not GABA_{A} receptors. In other embodiments, a GABA_{C} receptor antagonist inhibits (for example, statistically significantly inhibits) expression and/or activity of GABA_{C} receptors, but not GABA_{B} receptors. In still further embodiments, a GABA_{C} receptor antagonist inhibits (for example, statistically significantly inhibits) expression and/or activity of GABA_{C} receptors, but not GABA_{A} or GABA_{B} receptors. In one non-limiting example, a selective GABA_{C} receptor antagonist includes TPMPA. However, any compound that is a GABA_{C} receptor antagonist (for example, inhibits GABA_{C} receptor expression and/or activity) can be used in the disclosed methods.

It is to be understood that GABA_{C} receptor antagonists for use in the present disclosure include any known GABA_{C} receptor antagonists and also include novel GABA_{C} receptor antagonists developed in the future.

B. Metabotropic Glutamate Receptor Antagonists

In some embodiments, the mGluR receptor antagonists (for example, mGlu1 receptor antagonists) of use in the disclosed methods are small organic molecule antagonists. In some examples, a mGlu1 receptor antagonist includes 3,4-dihydro-2H-pyranol[2,3-b]quinolin-7-yl-(cis-4-methoxycyclohexyl)-methanone (JNJ16259685); 6-amino-N-cyclohexyl-N,3-dimethylthiazolo[3,2-a]benzimidazole-2-carboxamide hydrochloride (YM-298198); 4-[1-(2-fluoropyridin-3-yl)-5-methyl-1H-1,2,3-triazol-4-yl]-N-isopropyl-N-methyl-3,6-dihydropyridine-1(2H)-caroxamide (FTIDC); or 2-cyclopropyl-5-[1-(2-fluoro-3-pyridinyl)-5-methyl-1H1,2,3-triazol-4-yl]-2,3,-dihydro-1H-isoindol-1-one (CFMTI). In other examples, a mGlu1 receptor antagonist includes 7-(hydroxyimino)cyclopropa[b]chromen-1a-carboxylate ethyl ester (CPCCOEt); 1-aminoindan-1,5-dicarboxylic acid (AIDA); 3-Amino-6-chloro-5-dimethylamino-N-2-pyridinylpyrazinecarboxamide hydrochloride (ACDPP); DL-2-Amino-3-phosphonopropionic acid (DL-AP3); 9-(Diethylamino)-3-(hexahydro-1H-azepin-1-yl)pyrido[3′,2′:4,5]thieno[3,2-d]pyrimidin-4(3H)-one (A 841720) (3aS,6aS)-Hexahydro-5-methylene-6a-(2-naphthalenylmethyl)-1H-cyclopenta[c]furan-1-one (BAY 36-7620); N-(3-Chlorophenyl)-N′-(4,5-dihydro-1-methyl-4-oxo-1H-imidazol-2-yl)urea (Fenobam); (S)-4-carboxyphehylglycine (4 CPG); (S)-4-carboxy-3-hydroxyphenylglycine ((S)-4C3HPG); (S)-3-Carboxy-4-hydroxyphenylglycine ((S)-3C4HPG); (S)-(+)-α-Amino-4-carboxy-2-methylbenzeneacetic acid (LY 367385); 6-methoxy-N-(4-methoxyphenyl)quinazolin-4-amine hydrochloride (LY 456236 hydrochloride); α-Amino-5-carboxy-3-methyl-2-thiopheneacetic acid (3-MATIDA); α-methyl-4-carboxyphehylglycine (MCPG); (S)-α-Methyl-4-carboxyphenylglycine ((S)-MCPG); (RS)-α-Methyl-4-carboxyphenylglycine ((RS)-MCPG); 2-methyl-6-(phenylethynyl)-pyridine (MPEP); MPEP hydrochloride; 3-[(2-methyl-1,3-thiazol-4-yl)ethynyl]-pyridine (MTEP); N-Phenyl-7-(hydroxyimino)cyclopropa[b]chromen-1a-carboxamide (PHCCC); 6-Methyl-2-(phenylazo)-3-pyridinol (SIB 1757); 2-Methyl-6-(2-phenylethenyl)pyridine (SIB 1893); 6-amino-N-cyclohexyl-N,3-dimethylthiazolo[3,2-a]benzimidazole-2-carboxamide (YM 193167); N-tricyclo[3.3.1.13,7]dec-1-yl-2-quinoxalinecarboxamide (NPS 2390); 3-(5-(pyridin-2-yl)-2H-tetrazol-2-yl)benzonitrile; 3-[3-fluoro-5-(5-pyridin-2-yl-2H-tetrazol-2-yl)phenyl]-4-methylpyridine; 3-fluoro-5-(5-pyridin-2-yl-2-tetrazol-2-yl)benzonitrile; N-cyclohexyl-6-[[(2-methoxyethyl)-N-methylamino]methyl]-N-methylthiazolo[3,2-a]benzimidazole-2-carboxamide (YM 202074); 4-(Cycloheptylamino)-N-[[(2R)-tetrahydro-2-furanyl]methyl]-thieno[2,3-d]pyrimidine-6-methanamine (YM 230888); 6-Amino-N-cyclohexyl-3-methylthiazolo[3,2-a]benzimidazole-2-carboxamide hydrochloride (Desmethyl-YM298198); (RS)-α-Ethyl-4-carboxyphenylglycine, (E4CPG); α-Amino-4-hexyl-2,3-dihydro-3-oxo-5-isoxazolepropanoic acid (Hexylhomoibotenic acid; HexylHIBO); (S)-α-Amino-4-hexyl-2,3-dihydro-3-oxo-5-isoxazolepropanoic acid; ((αS)-Hexylhomoibotenic acid; (S)-HexylHIBO); 3-ethyl-2-methyl-quinolin-6-yl)-(4-methoxy-cyclohexyl)-methanone methanesulfonate (EMQMCM); 1-(3,4-dihydro-2H-pyrano[2,3-b]quinolin-7-yl)-2-phenyl-1-ethanone (R214127); 3,3′-Difluorobenzaldazine (DFB); [(3-Methoxyphenyl)methylene]hydrazone-3-methoxybenzaldehyde (DMeOB); (Diphenylacetyl)-carbamic acid ethyl ester (Ro 01-6128); (9-H-Xanthen-9-ylcarbonyl)-carbamic acid butyl ester (Ro 67-4853); (2S)-2-(4-Fluorophenyl)-1-[(4-methylphenyl)sulfonyl]-pyrrolidine, (Ro 67-7476). See, e.g., Lavreysen et al. (Mol. Pharmacol. 63:1082-1093, 2003); Lavreysen et al. (Neuropharmacol. 47:961-972, 2004); Satow et al. (J. Pharmacol. Exp. Ther. 330:179-190, 2009); Suzuki et al (J. Pharmacol. Exp. Ther. 321:1144-1153, 2007); Fukunaga et al. (Br. J. Pharmacol. 151:870-876, 2007); U.S. Pat. No. 7,989,464; U.S. Pat. App. Publ. No. 2011/0263652, all of which are incorporated herein by reference in their entirety. In one particular example, the mGlu1 receptor antagonist is JNJ16259685.

In some examples, a mGlu1 receptor antagonist has a structure represented by:

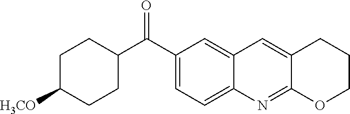

In other examples, a mGlu1 receptor has one of the following structures:

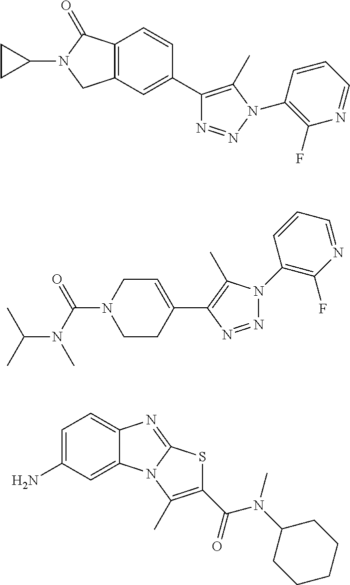 [SMILES: Cc1c(-c2ccc3c(c2)CN(C2CC2)C3=O)nnn1-c1cccnc1F ; Cc1c(C2=CCN(C(=O)N(C)C(C)C)CC2)nnn1-c1cccnc1F ; Cc1c(C(=O)N(C)C2CCCCC2)sc2nc3ccc(N)cc3n12]

In other embodiments, the mGlu1 receptor antagonist is an antisense compound. Any type of antisense compound that specifically targets and regulates expression of a mGlu1 receptor is contemplated for use. Methods of designing, preparing and using mGlu1 receptor antisense compounds are within the abilities of one of skill in the art, for example, utilizing publicly available mGlu1 receptor sequences. Antisense compounds specifically targeting mGlu1 receptor can be prepared by designing compounds that are complementary to a mGlu1 receptor nucleotide sequence, such as a mGlu1 receptor mRNA sequence. Antisense compounds need not be 100% complementary to the target nucleic acid molecule to specifically hybridize and regulate expression the target gene. For example, the antisense compound, or antisense strand of the compound if a double-stranded compound, can be at least 75%, at least 80%, at least 85%, at least 90%, at least 95%, at least 99%, or 100% complementary to the selected mGlu1 receptor nucleic acid sequence, such as about 20-25 contiguous nucleotides of a mGlu1 receptor nucleic acid. Particular examples of mGlu1 receptor nucleic acid sequences are provided above. Exemplary mGlu1 receptor antisense compounds are commercially available (for example, from Santa Cruz Biotechnologies (Santa Cruz, Calif.); or Thermo Scientific Dharmacon (Lafayette, Colo.)). Methods of screening antisense compounds for specificity are well known in the art.

In other embodiments, the mGlu1 receptor antagonist is an antibody. Any type of antisense compound that specifically binds and regulates activity of a mGlu1 receptor is contemplated for use. One of ordinary skill in the art can readily generate antibodies which specifically bind to a mGlu1 receptor. These antibodies can be monoclonal or polyclonal. They can be chimeric or humanized. Any functional fragment or derivative of an antibody can be used including Fab, Fab′, Fab2, Fab′2, and single chain variable regions. So long as the fragment or derivative retains specificity of binding for mGlu1 receptor it can be used in the methods provided herein. Antibodies can be tested for specificity of binding by comparing binding to appropriate antigen (e.g., a mGlu1 receptor or portion thereof) to binding to irrelevant antigen or antigen mixture under a given set of conditions. If the antibody binds to appropriate antigen at least 2, at least 5, at least 7, or 10 times more than to irrelevant antigen or antigen mixture, then it is considered to be specific. Exemplary mGlu1 receptor antibodies are commercially available (for example, from Santa Cruz Biotechnologies (Santa Cruz, Calif.); or Abcam (Cambridge, Mass.)).

In some embodiments, the mGlu1 receptor antagonist is a selective mGlu1 receptor antagonist, for example, a compound that inhibits activity or expression of a mGlu1 receptor, but does not inhibit (for example, does not statistically significantly inhibit) activity or expression of one or more other mGlu receptors (such as mGlu2, mGlu3, mGlu4, mGlu5, mGluR6, mGlu7, and/or mGlu8). In other examples, a mGlu1 receptor antagonist inhibits (for example, statistically significantly inhibits) expression and/or activity of mGlu1 receptor receptors, but not mGlu5 receptors (for example, is a selective mGlu1 antagonist). In one non-limiting example, a selective mGlu1 receptor antagonist includes JNJ16259685. However, any compound that is a mGlu1 receptor antagonist (for example, inhibits mGlu1 receptor expression and/or activity) can be used in the disclosed methods.

It is to be understood that mGlu1 receptor antagonists for use in the present disclosure include any known mGlu1 receptor antagonists and also include novel mGlu1 receptor antagonists developed in the future.

### IV. MODES OF ADMINISTRATION

Pharmaceutical compositions that include one or more of the inhibitors of GABA signaling disclosed herein (such as 2, 3, 4, 5, or more GABA_{C} and/or mGlu1 receptor antagonists) can be formulated with an appropriate solid or liquid carrier, depending upon the particular mode of administration chosen. The pharmaceutically acceptable carriers and excipients useful in this disclosure are conventional. See, e.g., Remington: The Science and Practice of Pharmacy, The University of the Sciences in Philadelphia, Editor, Lippincott, Williams, & Wilkins, Philadelphia, Pa., 21^{st} Edition (2005). For instance, parenteral formulations usually include injectable fluids that are pharmaceutically and physiologically acceptable fluid vehicles such as water, physiological saline, other balanced salt solutions, aqueous dextrose, glycerol or the like. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, pH buffering agents, or the like, for example sodium acetate or sorbitan monolaurate. Excipients that can be included are, for instance, proteins, such as human serum albumin or plasma preparations.

The dosage form of the pharmaceutical composition will be determined by the mode of administration chosen. For instance, in addition to injectable fluids, topical, inhalation, oral and intraocular formulations can be employed. Topical preparations can include eye drops, ointments, sprays, patches and the like. Inhalation preparations can be liquid (e.g., solutions or suspensions) and include mists, sprays and the like. Oral formulations can be liquid (e.g., syrups, solutions or suspensions), or solid (e.g., powders, pills, tablets, or capsules). For solid compositions, conventional non-toxic solid carriers can include pharmaceutical grades of mannitol, lactose, starch, or magnesium stearate. Actual methods of preparing such dosage forms are known, or will be apparent, to those skilled in the art.

In some examples, the pharmaceutical composition may be administered by any means that achieve their intended purpose. Amounts and regimens for the administration of the selected GABA_{C} or mGlu1 receptor antagonists will be determined by the attending clinician. Effective doses for therapeutic application will vary depending on the nature and severity of the condition to be treated, the particular compound(s) selected, the age and condition of the patient, and other clinical factors. Typically, the dose range will be from about 0.001 mg/kg body weight to about 500 mg/kg body weight. Other suitable ranges include doses of from about 0.01 mg/kg to 1 mg/kg, about 0.1 mg/kg to 30 mg/kg body weight, about 1 mg/kg to 100 mg/kg body weight, or about 10 mg/kg to about 50 mg/kg. The dosing schedule may vary from once a week to daily or multiple times per day, depending on a number of clinical factors, such as the subject's sensitivity to the compound. Examples of dosing schedules are about 1 mg/kg administered twice a week, three times a week or daily; a dose of about 10 mg/kg twice a week, three times a week or daily; or a dose of about 100 mg/kg twice a week, three times a week or daily.

The pharmaceutical compositions that include one or more of the disclosed inhibitors of GABA signaling can be formulated in unit dosage form, suitable for individual administration of precise dosages. In one specific, non-limiting example, a unit dosage can contain from about 1 ng to about 500 mg of a GABA_{C} or mGlu1 receptor antagonist (such as about 100 ng to 100 μg, about 1 ng to 1 μg, about 10 μg to 10 mg, about 1 mg to 100 mg or about 10 mg to 50 mg). The amount of active compound(s) administered will be dependent on the subject being treated, the severity of the affliction, and the manner of administration, and is best left to the judgment of the prescribing clinician. Within these bounds, the formulation to be administered will contain a quantity of the active component(s) in amounts effective to achieve the desired effect in the subject being treated. In some examples, the GABA_{C} or mGlu1 receptor antagonist is administered daily, weekly, bi-weekly, or monthly. In other examples, the GABA_{C} or mGlu1 receptor antagonist is administered one or more times a day, such as once, twice, three, or four times daily.

The compounds of this disclosure can be administered to humans or other animals on whose tissues they are effective in various manners such as topically, orally, intravenously, intramuscularly, intraperitoneally, intranasally, intradermally, intrathecally, subcutaneously, intraocularly, via inhalation, or via suppository. In one example, the compounds are administered to the subject topically. In another example, the compounds are administered to the subject intraocularly (for example intravitreally). In some examples, the amount of compound is sufficient to result in a vitreal concentration of about 1 nM to 500 μM (such as about 1 nM to 1 μM, about 10 nM to 100 nM, about 0.1 μM to about 250 μM, about 1 μM to about 200 μM or about 10 μM to about 100 μM). In further examples, the compounds are administered orally or intravenously. The particular mode of administration and the dosage regimen will be selected by the attending clinician, taking into account the particulars of the case (e.g., the particular GABA_{C} or mGlu1 receptor antagonist, the subject, the disease, the disease state involved, and whether the treatment is prophylactic). Treatment can involve monthly, bi-monthly, weekly, daily or multi-daily doses of compound(s) over a period of a few days to months, or even years.

In some embodiments, the disclosed GABA_{C} or mGlu1 receptor antagonists can be included in an inert matrix for either topical application or injection into the eye, such as for intravitreal administration. As one example of an inert matrix, liposomes may be prepared from dipalmitoyl phosphatidylcholine (DPPC), such as egg phosphatidylcholine (PC). Liposomes, including cationic and anionic liposomes, can be made using standard procedures as known to one skilled in the art. Liposomes including one or more GABA_{C} and/or mGlu1 receptor antagonists can be applied topically, either in the form of drops or as an aqueous based cream or gel, or can be injected intraocularly (such as by intravitreal injection). In a formulation for topical application, the compound is slowly released over time as the liposome capsule degrades due to wear and tear from the eye surface. In a formulation for intraocular injection, the liposome capsule degrades due to cellular digestion. Both of these formulations provide advantages of a slow release drug delivery system, allowing the subject to be exposed to a substantially constant concentration of the compound over time. In one example, the compound can be dissolved in an organic solvent such as DMSO or alcohol as previously described and contain a polyanhydride, poly(glycolic) acid, poly(lactic) acid, or polycaprolactone polymer.

The GABA_{C} or mGlu1 receptor antagonists can be included in a delivery system that can be implanted at various sites in the eye, depending on the size, shape and formulation of the implant, and the type of transplant procedure. The delivery system is then introduced into the eye. Suitable sites include but are not limited to the anterior chamber, anterior segment, posterior chamber, posterior segment, vitreous cavity, suprachoroidal space, subconjunctiva, episcleral, intracorneal, epicorneal and sclera. In one example, the delivery system is placed in the anterior chamber of the eye. In another example, the delivery system is placed in the vitreous cavity. In some examples, administering the GABA_{C} or mGlu1 receptor antagonist includes contacting the retina or cells of the retina (for example, one or more photoreceptors, bipolar cells, horizontal cells, or RGCs) with the antagonist.

In some examples, an effective amount of a GABA_{C} receptor antagonist can be the amount of a GABA_{C} receptor antagonist (such as TPMPA) necessary to increase responsiveness to light in a subject with retinal degeneration (such as RP or AMD). In some examples, an effective amount of a mGlu1 receptor antagonist can be the amount of a mGlu1 receptor antagonist (such as JNJ16259687, YM298198, CFMTI, or FTIDC) necessary to increase responsiveness to light in a subject with retinal degeneration (such as RP or AMD).

The present disclosure also includes combinations of one or more of the disclosed GABA_{C} and/or mGlu1 receptor antagonists with one or more other agents useful in the treatment of a retinal degeneration. For example, the compounds of this disclosure can be administered in combination with effective doses of one or more therapies for retinal disorders, including but not limited to, gene therapy (including optogenetic therapy), vitamin or mineral supplements (such as vitamins A, C, and/or E, or zinc and/or copper), anti-angiogenic therapy (such as ranibizumab or bevacizumab), photocoagulation, photodynamic therapy, lutein or zeaxanthin, corticosteroids, or immunosuppressants. Appropriate combination therapy for a particular disease can be selected by one of skill in the art. For example, the GABA_{C} and/or mGlu1 receptor antagonists of this disclosure can be administered in combination with an anti-angiogenic therapy, such as an anti-VEGF antibody (for example, bevacizumab or ranibizumab), an anti-VEGF nucleic acid (for example pegaptanib), or a VEGFR inhibitor (such as lapatinib, sunitinib, or sorafenib), to a subject with age-related macular degeneration. The term “administration in combination” or “co-administration” refers to both concurrent and sequential administration of the active agents.

The following examples are provided to illustrate certain particular features and/or embodiments. These examples should not be construed to limit the disclosure to the particular features or embodiments described.

### EXAMPLES

### Example 1

### Effect of TPMPA on Retinal Light Responsiveness

### Materials and Methods

Animals and Tissue Preparation:

Sprague-Dawley (SD) rats (age range 13-44 weeks) and P23H-line 1 homozygous rats (age range 23-42 weeks) were used in this study. Both the SD rats and P23H rats were bred and housed in the same facility. Breeding pairs of SD rats were obtained from Harlan Laboratories (Indianapolis, Ind.). Breeding pairs of P23H-line 1 homozygous rats were generously donated by Dr. Matthew LaVail (University of California San Francisco, Calif.). The room light was kept on a 12 hour light/dark cycle using standard fluorescent lighting. During the light cycle the illumination at the level of the cages was 100-200 lux. All animal care procedures and experimental methods were approved by the appropriate Institutional Animal Care and Use Committee.

On the day of an experiment, a rat was euthanized with sodium pentobarbital (150 mg/kg, i.p.), and the eyes were removed and hemisected under normal room light. After removal of the vitreous humour from each eye, one eyecup was transferred to a holding vessel containing bicarbonate-buffered Ames medium (Sigma-Aldrich, St. Louis, Mo.), which was continuously gassed at room temperature with 5% CO₂/95% O₂. The retina of the other eyecup was gently peeled from the choroid and trimmed into a square of about 12 mm². The retina was then placed photoreceptor side down in a small-volume (0.1 ml) chamber. The chamber was mounted on a fixed-stage upright microscope (Nikon Eclipse E600FN), and the retina superfused at 1.5 ml/min with bicarbonate-buffered Ames medium supplemented with 2 mg/ml D-(+) glucose and equilibrated with 5% CO₂/95% O₂. An in-line heating device (Warner Instruments, Hamden, Conn.) was used to maintain recording temperature at 35-36° C. The retina of the other eyecup was used later in the day.

Electrical Recording:

With the aid of red light (>630 nm) that was delivered from below the chamber, the tip of the recording microelectrode (0.7-1.3 MΩ impedance; Thomas Recording GmbH, Germany) was visually advanced to the retinal surface with a motor-driven micromanipulator. Extracellular potentials from retinal ganglion cells (RGCs) were amplified and bandpass filtered at 100 to 5000 Hz by a differential amplifier (Xcell-3; FHC, Bowdoin, Me.). To ensure that recordings were made from single cells, the recorded waveform of the action potential (spike) was continuously displayed in real time on a PC to check for uniformity of spike size and shape. Spikes from single RGCs were converted to standard transistor to transistor logic (TTL) pulses with a time-amplitude window discriminator (APM Neural Spike Discriminator, FHC). A laboratory data acquisition system (1401 Processor and Spike2 software; Cambridge Electronic Design Ltd., Cambridge, UK) was used to digitize the TTL pulses and raw spike train data.

Light Stimulation:

Light from a mercury arc lamp illuminated an aperture that was focused on the retina from above through the 4× objective of the microscope. The image produced on the retina was either a 250-μm or 1.5-mm diameter spot, which was centered on the recorded RGC. In the light path was an interference filter (peak transmission at 545 nm). The intensity of the unattenuated light stimulus on the retina measured with a spectroradiometer (ILT900-R, International Light Technologies, Peabody, Mass.) was 8.5×10¹⁷ photons/cm²/s. Neutral density filters were inserted in the light path to reduce the intensity of light stimulus. An electromechanical shutter (Uniblitz, Rochester, N.Y.) was used to control the stimulus duration, which was set to 100 msec in constructing intensity-response curves. During recordings from RGCs, light flashes were presented with interstimulus intervals of 3-6 sec to avoid any adapting effect of the previous flash. RGCs were classified as either ON-center or OFF-center from their response to a long duration (0.7-1.0 sec) flash. Experiments were performed in a dimly lighted room (10 lux).

Drug Application:

The GABA_{C} receptor antagonist (1,2,5,6-tetrahydropyridin-4-yl)methylphosphinic acid (TPMPA) was purchased from Sigma-Aldrich (St. Louis, Mo.). TPMPA was dissolved in saline solution at 10 mM and applied at a steady rate (via a syringe pump) to the bathing solution via the input line of the recording chamber. TPMPA was bath applied for about 10 minutes to achieve stable responses before its effects were tested. The effects of TPMPA were studied on only one RGC per retina.

Data Analysis:

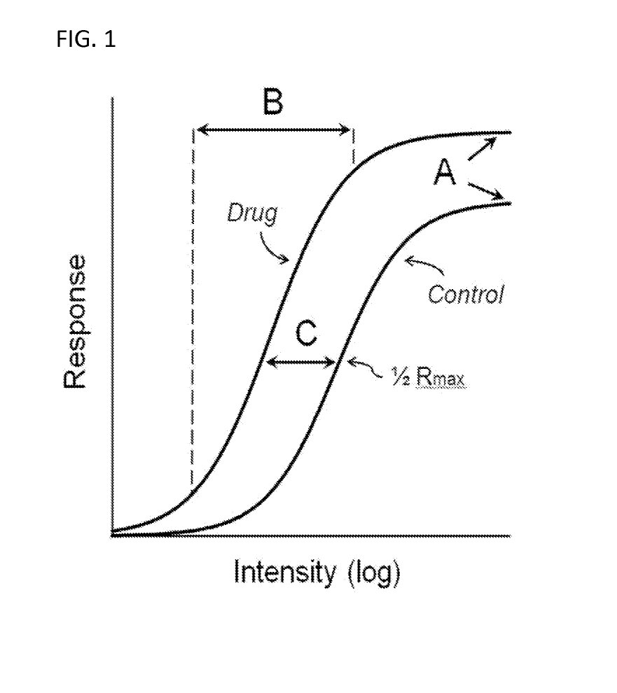

The light-evoked responses of RGCs were calculated by counting the number of spikes within a 100 msec window that encompassed the peak response and subtracting any baseline (spontaneous) activity measured between light stimuli. Cell responses were averaged from 5 stimulus presentations. Intensity-response curves of RGCs were fitted with a sigmoidal dose-response (variable slope), using SigmaPlot 10.0 (Systat Software, San Jose, Calif.). As illustrated in FIG. 1, three parameters were measured from the curve fits: maximum peak response, dynamic operating range, and light sensitivity. Data are expressed as mean±standard deviation. Statistical significance was assessed using paired Student's t-test, with P<-0.05 considered significant.

### Results

Data were collected on 27 P23H rat RGCs that were stimulated with either a 250-μm or 1.5-mm diameter spot of light centered over the receptive field. Fourteen RGCs were stimulated with the small spot of light; 13 RGCs were stimulated with the large spot of light. Since the outcomes of large and small spots of light did not reveal significant differences, data from both spots were pooled in the overall analysis. Of the 27 RGCs, 21 were ON-center cells and 6 were OFF-center cells.

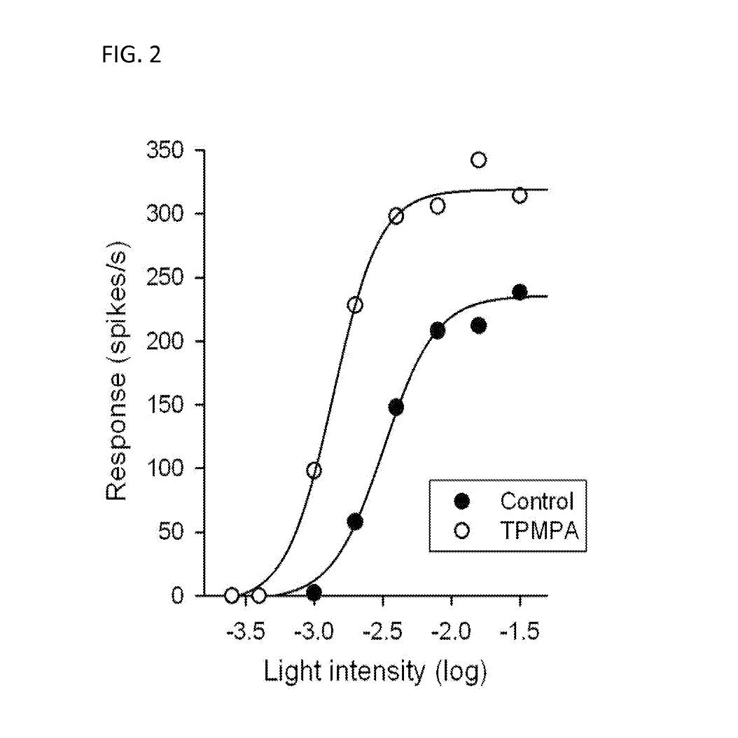

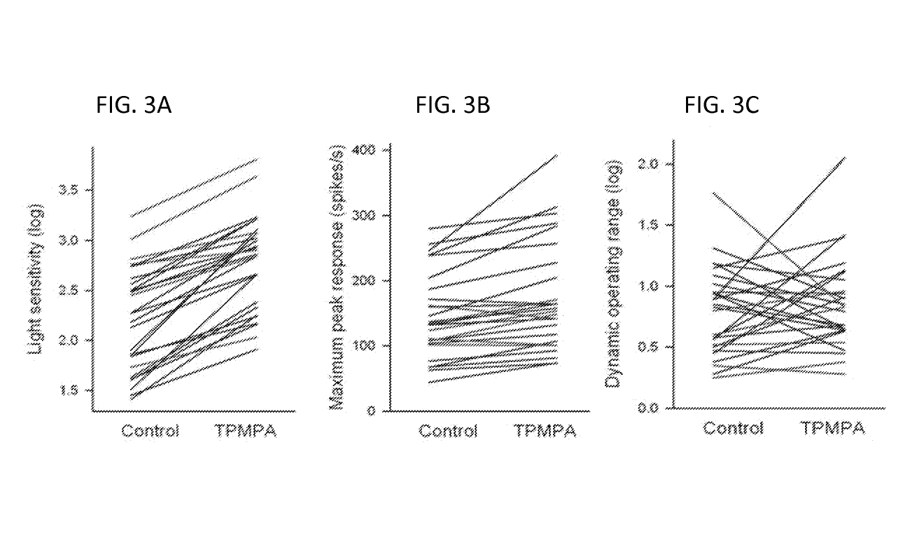

FIG. 2 shows the effect of TPMPA on a representative P23H rat RGC, which was an ON-center cell. The light intensity that evoked a half-maximum response prior to application of TPMPA was −2.48 log units attenuation. With application of TPMPA, the light intensity that evoked the same response was −2.94 log units attenuation. Therefore, TPMPA increased the sensitivity of this cell to light by 0.46 log unit. TPMPA increased the sensitivity of all 27 RGCs tested (FIG. 3A). For ON-center RGCs, the light intensity that generated a half-maximal response prior to application of TPMPA was on average −2.08±0.45 log units attenuation. In the presence of TPMPA, the same light-evoked response was obtained at a light intensity of −2.71±0.41 log units attenuation (0.63 log unit lower intensity). The difference of the means was statistically significant (P<0.001; paired t-test). For OFF-center RGCs, the light intensity that generated a half-maximal response prior to application of TPMPA was on average −2.67±0.53 log units attenuation. In the presence of TPMPA, the same light-evoked response was obtained at a light intensity of −3.05±0.64 log units attenuation (0.38 log unit lower intensity). The difference of the means was statistically significant (P=0.004; paired t-test).

FIG. 2 also shows that TPMPA increased the peak response of the cell to a high intensity light stimulus. The maximum peak response increased from 240 to 313 spikes/s. TPMPA increased the maximum peak response of 23 of 27 P23H rat RGCs to a high intensity light stimulus (FIG. 3B). For one ON-center cell and three OFF-center cells, the maximum peak response decreased slightly, on average by 6.7% (range: 1-12%). For all ON-center cells (n=21), the maximum peak response prior to application of TPMPA was on average 152±71 spikes/s. With application of TPMPA, the maximum peak response increased to 185±90 spikes/s. This 22% increase was statistically significant (P<0.001; paired t-test). For all OFF-center cells (n=6), the maximum peak response prior to application of TPMPA was on average 123±22 spikes/s. With application of TPMPA, the maximum peak response increased to 136±29 spikes/s. This 11% increase was not statistically significant (P=0.284; paired t-test).

FIG. 3C shows the effect of TPMPA on the dynamic operating range of ON-center and OFF-center P23H rat RGCs. The dynamic operating range of ON-center cells increased on average from 0.81±0.30 log unit (before application of TPMPA) to 0.92±0.38 log unit with application of TPMPA. This 14% increase in the dynamic operating range was not statistically significant (P=0.298; paired t-test). The dynamic operating range of OFF-center cells decreased on average from 0.71±0.43 log unit prior to application of TPMPA to 0.64±0.28 log unit with application of TPMPA. This 10% decrease in the dynamic operating range was not statistically significant (P=0.681; paired t-test).

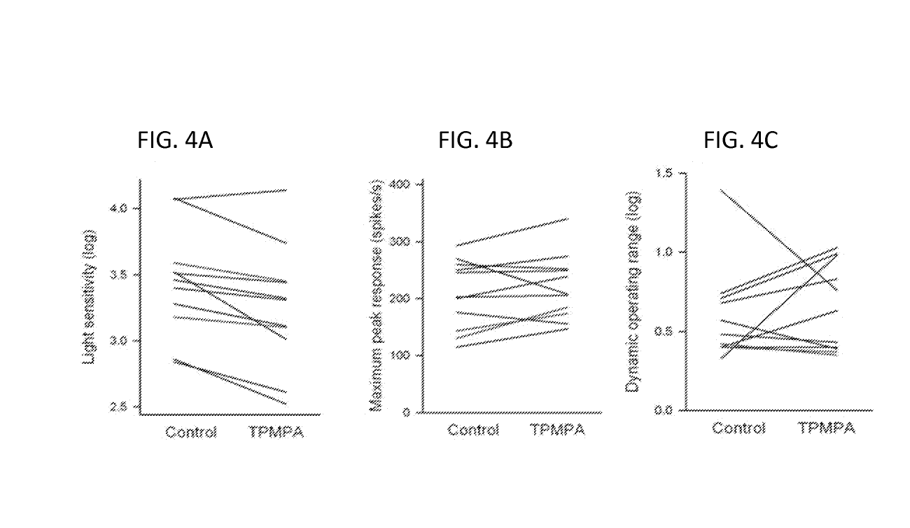

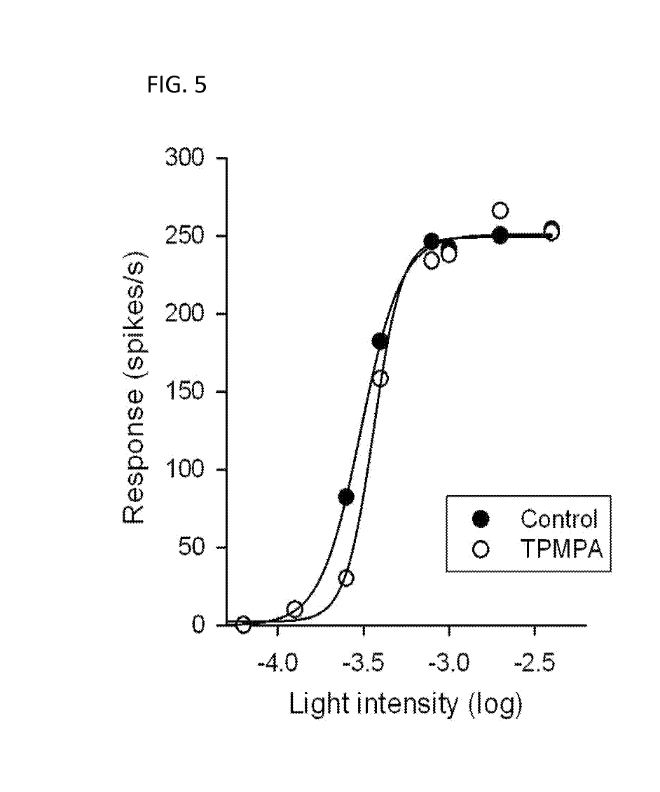

The effects of TPMPA were studied on 9 ON-center SD rat RGCs and 2 OFF-center SD rat RGCs. TPMPA did not increase the sensitivity of these cells to light, in contrast to the increase in sensitivity observed for P23H rat RGCs. On the contrary, TPMPA decreased light sensitivity of most cells (FIG. 4A). Except for one ON-center RGC, which exhibited a 0.07 log unit increase in light sensitivity, all other RGCs showed a decrease in light sensitivity in the presence of TPMPA. FIG. 5 shows the effect of TPMPA on a representative cell, which was an ON-center cell. The light intensity that generated a half-maximal response for this cell was −3.51 log units attenuation. In the presence of TPMPA, the same light-evoked response was obtained at a light intensity of −3.44 log units attenuation (0.07 log unit higher intensity). TPMPA decreased the response magnitude of this cell to a high intensity light stimulus from 260 to 252 spikes/s, and decreased the dynamic operating range from 0.57 to 0.39 log unit.

The light intensity that generated a half-maximal response for the ON-center SD rat RGCs (n=9) was on average −3.44±0.44 log units attenuation. In the presence of TPMPA, the same light-evoked response was obtained at a light intensity of −3.24±0.51 log units attenuation (0.20 log unit higher intensity). The difference of the means was statistically significant (P=0.008; paired t-test). TPMPA had very little effect on either the maximum peak response (FIG. 4B) or the dynamic operating range (FIG. 4C) of ON-center SD rat RGCs. On average the maximum peak response prior to application of TPMPA was 224±55 spikes/s. With application of TPMPA, the maximum peak response increased to 230±60 spikes/s. The difference of the means was not statistically significant (P=0.586; paired t-test). On average the dynamic operating range of ON-center SD rat RGCs increased from 0.63±0.32 log unit to 0.72±0.26 log unit with application of TPMPA. This 14% increase in the dynamic operating range was not statistically significant (P=0.512; paired t-test). For the two OFF-center SD rat RGCs studied, TPMPA reduced the sensitivity to light flashes by 0.08 and 0.14 log units and increased the maximum peak responses. The dynamic operating ranges were reduced only slightly.

### Example 2

### Effect of JNJ16259685 on Retinal Light Responsiveness

Experiments similar to those described in Example 1 were carried out on retinas isolated from P23H rats. The mGlu1 receptor antagonist 3,4-dihydro-2H-pyranol[2,3-b]quinolin-7-yl-(cis-4-methoxycyclohexyl)-methanone (JNJ16259685) was purchased from Tocris Bioscience (Bristol, UK). JNJ16259685 was dissolved in saline solution containing 0.01% dimethyl sulfoxide at 50 nM and applied at a steady rate (via a syringe pump) to the bathing solution via the input line of the recording chamber. Light responsiveness was measured as described in Example 1.

Light responsiveness of 16 RGCs (15 ON-center RGCs and one OFF-center RGC) from P23H rats was determined before and after application of JNJ16259685. Application of 500 nM JNJ16259685 to the retina increased light sensitivity on average by 0.56 log units, that is, in the presence of JNJ16259685, the cells responded to light that was almost 4-fold less intense than in the absence of JNJ16259685. JNJ16259685 increased the maximum peak response of RGCs from 162±65 spikes/s to 178±64 spikes/s. This 9.9% increase was statistically significant (P=0.007; paired t-test). Overall, JNJ16259685 increased the dynamic operating range of the RGCs from 0.89±0.41 log unit to 1.05±0.42 log unit. This 18% increase was not statistically significant (P=0.239; paired t-test).

### Example 3

### Methods of Increasing Retinal Responsiveness to Light in a Subject with a GABA_{C} Receptor Antagonist

This example describes exemplary methods for increasing retinal responsiveness to light in a subject with retinal degeneration. One of skill in the art will appreciate that methods that deviate from these specific methods can also be used to increase retinal responsiveness to light in a subject.

Subjects having a retinal degeneration (such as RP or AMD) are selected. In some cases, subjects are treated with an intravitreal sustained-release implant with (1,2,5,6-tetrahydropyridin-4-yl)methylphosphinic acid (TPMPA) at a vitreal concentration of about 0.1 μM to 200 μM. In other cases, subjects receive intraocular injections of about 100 ng to 100 μg TPMPA one to three times per week. Subjects are assessed for measures of visual or retinal function (such as visual acuity, visual field, electroretinogram, OCT, Amsler grid, fundus examination, color vision test, or fluorescein angiography), prior to initiation of therapy, periodically during the period of therapy, and/or at the end of the course of treatment. Subjects are also tested for retinal responsiveness to light (such as detectable light intensity threshold or frequency or magnitude of response to light), prior to initiation of therapy, periodically during the period of therapy and/or at the end of the course of treatment.

The effectiveness of TPMPA therapy to treat increase retinal light responsiveness in a subject can be demonstrated by an decrease in detectable light intensity threshold or an increase in frequency or magnitude of light response, for example, compared to a control, such as an untreated subject, a subject with retinal degeneration prior to treatment (for example, the same subject prior to treatment), or a subject with the same retinal degeneration treated with placebo (e.g., vehicle only).

### Example 4

### Methods of Increasing Retinal Responsiveness to Light in a Subject with a mGlu1 Receptor Antagonist

This example describes exemplary methods for increasing retinal responsiveness to light in a subject with retinal degeneration. One of skill in the art will appreciate that methods that deviate from these specific methods can also be used to increase retinal responsiveness to light in a subject.

Subjects having a retinal degeneration (such as RP or AMD) are selected. In some cases, subjects are treated with an intravitreal sustained-release implant with 3,4-dihydro-2H-pyranol[2,3-b]quinolin-7-yl-(cis-4-methoxycyclohexyl)-methanone (JNJ16259685) at a vitreal concentration of about 1 nM to 1 μM. In other cases, subjects receive intraocular injections of about 1 ng to 1 μg JNJ16259685 one to three times per week. Subjects are assessed for measures of visual or retinal function (such as visual acuity, visual field, electroretinogram, OCT, Amsler grid, fundus examination, color vision test, or fluorescein angiography), prior to initiation of therapy, periodically during the period of therapy, and/or at the end of the course of treatment. Subjects are also tested for retinal responsiveness to light (such as detectable light intensity threshold or frequency or magnitude of response to light), prior to initiation of therapy, periodically during the period of therapy and/or at the end of the course of treatment.

The effectiveness of JNJ16259685 therapy to treat increase retinal light responsiveness in a subject can be demonstrated by an decrease in detectable light intensity threshold or an increase in frequency or magnitude of light response, for example, compared to a control, such as an untreated subject, a subject with retinal degeneration prior to treatment (for example, the same subject prior to treatment), or a subject with the same retinal degeneration treated with placebo (e.g., vehicle only).

In view of the many possible embodiments to which the principles of the disclosure may be applied, it should be recognized that the illustrated embodiments are only examples and should not be taken as limiting the scope of the invention. Rather, the scope of the invention is defined by the following claims. We therefore claim as our invention all that comes within the scope and spirit of these claims.

1. A method of increasing retinal responsiveness to light in a subject with retinal degeneration, comprising:
selecting a subject with retinal degeneration; and
administering an effective amount of an inhibitor of retinal GABA signaling, thereby increasing the retinal responsiveness to light in the subject with retinal degeneration.

2. The method of claim 1, further comprising determining the retinal responsiveness to light in the subject.

3. The method of claim 2, wherein the retinal responsiveness to light in the subject is increased as compared to a control.

4. The method of claim 1, wherein the retinal degeneration comprises retinitis pigmentosa or macular degeneration.

5. The method of claim 1, wherein the inhibitor of GABA signaling is administered intraocularly or topically.

6. The method of claim 1, wherein administering the inhibitor of GABA signaling to the subject comprises contacting a retina of the subject with the inhibitor of GABA signaling.

7. The method of claim 6, wherein administering the inhibitor of GABA signaling to the subject comprises contacting at least one retinal ganglion cell or at least one bipolar cell of the subject with the inhibitor of GABA signaling.

8. The method of claim 1, wherein the retinal responsiveness to light comprises magnitude or sensitivity of response to a stimulus, or a combination thereof.

9. The method of claim 8, wherein the retinal responsiveness to light comprises the magnitude of response, wherein an increased magnitude of response comprises an increase in number, size, dynamic operating range or frequency of an electrical response by the retina to a stimulus, or a combination thereof.

10. The method of claim 8, wherein the retinal responsiveness to light comprises the sensitivity of response, wherein an increased sensitivity of response comprises a decrease in a threshold for response to a stimulus.

11. The method of claim 8, wherein the stimulus comprises a light stimulus, an electrical stimulus, or a combination thereof.

12. The method of claim 1, wherein retinal responsiveness to light is measured by direct electrical recording, electroretinogram, or visual evoked potential.

13. The method of claim 1, wherein the inhibitor of GABA signaling comprises a GABA_{C} receptor antagonist.

14. The method of claim 13, wherein the GABA_{C} receptor antagonist selectively inhibits a GABA_{C} receptor as compared to a GABA_{A} receptor.

15. The method of claim 13, wherein the GABA_{C} receptor antagonist comprises a small molecule, an antisense compound, or an antibody.

16. The method of claim 13, wherein the GABA_{C} receptor antagonist comprises 1,2,5,6,-(tetrahydropyridin-4-yl)methylphosphinic acid (TPMPA).

17. The method of claim 1, wherein the inhibitor of GABA signaling comprises a metabotropic glutamate receptor type 1 (mGlu1) antagonist.

18. The method of claim 17, wherein the mGlu1 receptor antagonist selectively inhibits a mGlu1 receptor as compared to a mGlu5 receptor.

19. The method of claim 17, wherein the mGlu1 receptor antagonist comprises a small molecule, an antisense compound, or an antibody.

20. The method of claim 17, wherein the mGlu1 receptor antagonist comprises 3,4-dihydro-2H-pyranol[2,3-b]quinolin-7-yl-(cis-4-methoxycyclohexyl)-methanone (JNJ16259685).

21. The method of claim 1, further comprising administering to the subject an effective amount of a second therapeutic agent for retinal degeneration.

22. A method of increasing retinal responsiveness to light in a subject with retinal degeneration, comprising:
selecting a subject with retinal degeneration; and
administering an effective amount of 1,2,5,6,-(tetrahydropyridin-4-yl)methylphosphinic acid to the subject, thereby increasing the retinal responsiveness to light in the subject with retinal degeneration.

23. A method of increasing retinal responsiveness to light in a subject with retinal degeneration, comprising:
selecting a subject with retinal degeneration; and
administering an effective amount of 3,4-dihydro-2H-pyranol[2,3-b]quinolin-7-yl-(cis-4-methoxycyclohexyl)-methanone (JNJ16259685) to the subject, thereby increasing the retinal responsiveness to light in the subject with retinal degeneration.
